(19) United States
(12) Patent Application Publication
(10) Pub. No.: US 2008/0032377 A1
(43) Pub. Date: Feb. 7, 2008

(54) Uracil-DNA nuclease: protein enzyme possessing nuclease activity specific for uracil containing nucleic acid, process for its preparation and methods of use

(76) Inventors: Beata Grolmuszne Vertessy, Budapest (HU); Angela Beke, Homokszentgyorgy (HU); Ferenc Felfoldi, Budapest (HU); Vince I Grolmusz, Budapest (HU); Maria Pukancsik, Budapest (HU)

Correspondence Address: VINCE GROLMUSZ, UGRON GABOR U.8., BUDAPEST, 1118, omitted

(21) Appl. No.: 11/160,040

(22) Filed: Jun. 7, 2005

Related U.S. Application Data
(60) Provisional application No. 60/521,645, filed on Jun. 10, 2004.

(51) Int. Cl. C12N 9/22

(52) U.S. Cl. 435/199

(57) ABSTRACT

A uracil-specific endonuclease enzyme is discovered in specific developmental stages of Drosophila melanogaster. The protein responsible for this enzymatic activity has been isolated, cloned, sequenced, and expressed to practical homogeneity. Sequence homologues have been identified from four additonal organisms. The activity of this enzyme is strictly specific for uracil-containing DNA and persists in the presence of 1 mM concentration of divalent metal ion chelating agent ethylenediaminetetraacetic acid. The unique specificity and the observed metal-ion independent characteristics of this enzymatic activity allow its use for specific degradation of uracil-DNA in a simple enzymatic process while normal DNA is uncleaved by the enzyme. Claims of the present invention concern the enzyme, its homologues, its production, as well as its use for specific degradation of uracil-DNA in any application it might occur, all based on the discovery of the novel uracil-specific nuclease enzyme.

The invention concerns a novel enzyme protein capable of specific cleavage and degradation of uracil-DNA while it does not cleave normal DNA, its enzymatic activity being also present in the presence of 1 mM concentration of divalent metal ion chelating agent ethylenediaminetetraacetic acid; the protein homologues of this novel enzyme from four additional species; recombinant vectors comprising the nucleic acid sequence of the novel uracil-DNA nuclease protein; methods for expression and purification of this novel uracil-DNA enzyme protein; and methods for using the uracil-DNA nuclease enzyme protein to degrade uracil-DNA.

For the use within the present invention, uracil-DNA is defined as a circular or linear double stranded DNA species of arbitrary molecular weight that contains an arbitrary number, but larger than one, of deoxyuridine residues replacing thymine residues.

The chemical building blocks of DNA are as follows: deoxyribose, phosphate group, and bases that are linked by N-glycosidic bond to the deoxyribose. The deoxyribose and the phosphate group together form the sugar-phosphate backbone. Generally, only four bases are allowed to occur in DNA: adenine, thymine, guanine and cytosine. The sequence of these bases constitute the genetic code, that is represented in both strands of the DNA by the hydrogen-bonded pairing of the complemantary bases:adenine-thymine, guanine-cytosine. In RNA, that is chemically very similar to DNA, thymine is replaced by uracil. Uracil is an equally perfect hydrogen-bonding partner for adenine as thymine: in both adenine-thymoine and adenine-uracil base pairs the same hydrogen-bonds are formed (Watson-Crick base pairing). The only difference between uracil and thymine is the presence of a methyl group at the C-5 location in the pyrimidine ring that is absent from uracil.

The equivalence of thymine and uracil in forming Watson-Crick base pairs with adenine puts forward an intriguing question. Namely, why do living organisms require both thymine and uracil, why is uracil not enough to fulfill the role of the adenine base pair? It is also important to consider that thymine (thymidylate) biosynsthesis constitutes a significant metabolic task: the addition of the methyl group on the uracil base requires the presence and fine regulation of at least two further enzymes (thymidylate synthase and dihydrofolate reductase). Current experimental data indicate that the requirement for thymine is due to the chemical reactivity of cytosine that is the Watson-Crick base pair of guanine (Lindahl, T. (1993) Nature 362, 709-715). Cytosine is spontaneously deaminated to produce uracil: several hundreds of such deamination events occur per day in an average-sized mammalian genome under normal physiological conditions. The uracil bases that are produced in these deamination reactions will base pair with adenine in the next DNA replication cycle. Therefore, deaminated cytosine induces a stable point mutation: the exchange of a cytosine-guanine base pair into a uracil-adenine base pair. In order to prevent this kind of mutation, a DNA-repair system was developed during evolution (base-excision repair) that cuts out uracil from DNA, and reintroduces the correct base.

The base-excision repair system cuts out adenine-pairing uracil bases as well. It is therefore necessary to distinguish uracil bases that form base pairs with adenine and uracil bases that have been produced by the oxidative deamination of cytosine. The distinctive label was adopted on the form of a methyl group that became introduced into the pyrimidine ring of uracil to produce thymine. The introduction of the methyl group did not perturb the base pairing functions towards adenine, but it resulted in straightforward distinction from uracil (aka deaminated cytosine). The methyl label was introduced only in the DNA molceules responsible for long-term information storage, but not in the usually short-live dRNA molecules. The different life-times of DNA as compared to RNA results in much less cytosine deamination events occurring in RNA, rendering the distinction between adenine-pairing uracil and deaminated cytosines unnecessary.

In complex organisms, several protein-DNA interactions also rely on the distinctive function of the methyl label on thymine (Plaxco, K. W., and Goddard, W. A., 3rd. (1994) Biochemistry 33, 3050-3054). Although these recognition systems play an important regulatory role (e.g. in promoter regions to modulate gene expression), the regulations are important only in in vivo systems and in functions separate from replicative or repair DNA synthesis per se. During in vitro DNA synthesis (e.g. polymerase chain reaction, PCR), in the presence of adequeate nucleotide triphosphates (dATP, dGTP, dCTP and dUTP, in the absence of dTTP) polymerases will introduce uracil to face adenine. The synthesized uracil-DNA is equally capable to store genetic information, just like the original thymine-DNA. Using either uracil-DNA or thymine-DNA as a template, the same RNA molecules will be synthesized. In addition, in the presence of dTTP and the other three nucleotide triphosphates (dCTP, dATP, and dGTP), uracil-DNA can be used as a template for DNA polymerases to get back the original thymine-DNA with the same sequence (i.e. genetic) information.

In addition to the in vitro data, the physiological competence of uracil-DNA is further supported by results indicating the possibility of extensive thymine-uracil replacement in E. coli strains. In double null-mutant dut-ung-E. coli strains, where both enzymes sensing uracil as a mistake in DNA, i.e. uracil-DNA glycosylase and dUTPase are lacking, uracil-DNA is synthesized under physiological conditions (Warner H R and Duncan B K et al (1978) Nature 272:32-4, Warner H R et al (1981) J Bacteriol. 145:687-95, el-Hajj, H H et al (1992) J. Bacteriol. 174:4450-6).

These strains are as fit and viable as wild type, only their mutation rates are higher. The increased mutation rates are caused by lack of base-excision repair for the deaminted cytosine (aka uracil) bases, due to the absence of the enzyme uracil-DNA glycosidase. In E. coli, however, increased mutation rates do not induce loss of viability.

In conclusion, ample evidence indicates that substition of thymine by uracil in DNA does not change genetic information. Uracil-DNA is equivalent in all coding aspects to thymine-DNA in both in vitro, and in simple bacterial systems.

It was recently observed that extracts of Drosophila melanogaster third instar larvae are associated with a nuclease activity strictly specific for uracil-containing DNA. This enzyme was named uracil-DNA nuclease after its unique specificity. The enzymic activity, in contrast to most nucleases, did not require the presence of divalent cations. Recently, a metal-ion independent apoptotic nuclease was described in several organisms, this activity, however, was not uracil-DNA specific (Evans C J et al (2002) Gene 295:61-70). The newly discovered activity, however, cannot be observed on thymine-containing DNA. This novel uracil-DNA nuclease enzyme protein, and its uses as a specific cleavage tool for uracil-DNA constitute the present invention.

No enzyme with such a uracil-DNA specific nuclease activity has been identified till present. The finding is therefore possess significant novelty. The uracil-DNA nuclease is unique in its specificity that seems to be independent from divalent cations and can be observe up to 1 mM concentrations of divalent metal ion chelating agent ethylenediaminetetraacetic acid.

Identification of the protein responsible for uracil-DNA enzymatic activity in Drosophila melanogaster. Considering the uracil-specificity of the enzymatic activity identified in Drosophila extracts, a procedure was designed for identification of the protein responsible for this activity. Extracts from Drosophila larvae were passed through an affinity column upon which either normal DNA or uracil-DNA was immobilized. Following several washes, bound fractions have been eluted with increasing salt (sodium-chloride) concentration. Eluted fractions were analyzed by sodium-dodecyl sulfate polyacrylamide gel electrophoresis. Bands on the gel indicated proteins that were capable of binding to either normal DNA, or uracil-DNA, or both. Bands were classified according to apparent molecular weight positions. A protein band at approximately 40 kDa apparent molecular weight showed significant specific binding to uracil-DNA. It was analyzed by mass spectrometry. The mass spectrometric analysis identified this protein as the gene product of the gene CG18410 of Drosophila melanogaster. This gene has no identified function in the Drosophila genome database.

The coding sequence of this protein was found in Drosophila melanogaster samples of different developmental stages as transcribed into mature messenger RNA by reverse transcription of the total mRNA pool into cDNA followed polymerase chain reaction with adequate primer oligonucleotides. The cDNA coding sequence corresponding to SEQ ID NO:6 was cloned into expression vectors as follows:

It was subcloned into vector pET22b (Novagen, EMD Biosciences, Darmstadt, Germany), using EcoRI-Xhol restriction sites originating in plasmid PETUDE. The coding sequence for a maltose binding protein was excised from plasmid pMal-c2E (New England BioLabs, Schwalbach, Germany) by Ndel-BamHI digestion, and was subcloned into plasmid PETUDE using Ndel-BamHI restriction sites originating in plasmid pETMalUDE. In plasmid pETMalUDE, the coding sequence of uracil-DNA endonuclease is at the 3′-end to the coding sequence of the maltose-binding protein. The nucleic acid segment encoding the uracil-DNA endonuclease coding sequence was also subcloned into vector PET19b (Stratagene), using Ndel-Xhol restriction enzyme sites originating in plasmid pETHisUDE. In pETHisUDE, the coding sequence of uracil-DNA endonuclease is at the 3′-end to the coding sequence of the ten-histidine affinity tag.

The expression vectors pETMalUDE and pETHisUDE were used to transform E. coli cells. Expression of the uracil-DNA nuclease protein with the affinity tag maltose binding protein or polyhistidine was performed by iso-propyl-thio-galactoside induction following general methods known to one skilled in the art. It will be evident to one skilled in the art that similar procedures without the exercise of inventive skill may easily result in expression vectors also containing the coding sequence of uracil-DNA nuclease. either with an affinity tag or without such tag. Such vectors may also be used for expression of the protein uracil-DNA nuclease using methods known in the art. Such equivalents are intended to be encompassed by the following claims.

Due to the known degeneracy of the genetic code, it will be also apparent to those skilled in the art that different, but equivalent nucleotide sequences which code for the uracil-DNA nuclease enzyme of the invention, as shown in SEQ ID NO:1, or SEQ ID NO:2, or SEQ ID NO:3, or SEQ ID NO:4, or SEQ ID NO:5, may be isolated, synthesized or otherwise prepared without the exercise of the inventive skill. Such degenerate and equivalent coding sequences are included within the scope of the present invention.

Uracil-DNA nuclease activity of the recombinant uracil-DNA nuclease protein. The protein was expressed as described above. It was isolated from E. coli cell lysate and purified using the affinity tag with usual methods known in the art. Its activity was tested on uracil-DNA and normal DNA. Uracil-DNA was produced in the form of a plasmid isolated from dut-ung-double mutant E. coli cells. Normal DNA was produced in the form of plasmid from wild type E. coli cells. Plasmid preparations were according to usual methods known in the art. The recombinant protein, purified to approximataly 98% homogeneity, and containing the amino acid sequence constituted in SEQ ID NO:1 was incubated with normal DNA and uracil-DNA at 37.degree.C. for 10, 30, and 60 minutes in a solution containing 10 micrograms/ml uracil-DNA nuclease, 20 micrograms/ml DNA, 25 mmole/liter Hepes buffer, 150 mmole/liter sodium-chloride. Uracil-DNA was fragmented into small oligonucleotides while normal DNA showed practically no fragmentation at all. It was concluded that the protein enzyme uracil-DNA nuclease is strictly specific for uracil-DNA and is capable of fragmenting it into smaller oligonucleotides.

DNA binding affinity of recombinant uracil-DNA nuclease. The DNA binding affinity was tested on gel shift assay using usual methods known in the art. The recombinant uracil-DNA nuclease was shown to bind to uracil-DNA and normal DNA with comparable affinities. It was concluded that uracil-DNA nuclease has a general DNA binding ability which is not strictly specific for uracil-DNA.

Homologues of uracil-DNA nuclease. Homologue sequences of uracil-DNA nuclease from Drosophila melanogaster as contained in the amino acid sequence constituted in SEQ ID NO:1 or as contained in the nucleotide sequence constituted in SEQ ID No:6 were used to search for homologues in the usual databases known in the art. Four such homologues were identified, as contained in the amino acid sequence constituted in SEQ ID NO:2, or SEQ ID NO:3, or SEQ ID NO:4, SEQ ID NO:5, or as contained in the nucleotide sequence constituted in SEQ ID No:7, or SEQ ID No:8, or SEQ ID No:9, or SEQ ID No:10. All these homologues are present in genomes of metamorphing insects. It is concluded that the sequences contained in amino acid sequences constituted in SEQ ID NO:1, or SEQ ID NO:2, or SEQ ID NO:3, or SEQ ID NO:4, SEQ ID NO:5. correspond to proteins specific to metamorphing insects and other organisms may only encode such distant relatives that are not evident at the present.

Novelty of the uracil-DNA nuclease enzyme protein. The protein as contained in the amino acid sequence constituted in SEQ ID NO:1, or SEQ ID NO:2, or SEQ ID NO:3, or SEQ ID NO:4, SEQ ID NO:5. does not show significant homology to any of the known nucleases. Its active site may therefore constitute novel characteristics, exploitable in molecular biology application. Such an example may be that the enzymatic activity of uracil-DNA nuclease protein which is the subject of the present invention does not require the presence of divalent metal ions. This characteristics makes this enzyme a useful and rather unique tool for any molecular biology, or other applications where divalent metal ion-independent nuclease activity is used. Such applications are intended to be encompassed in the present invention.

Methods of use of uracil-DNA nuclease. The uracil-DNA nuclease of the present invention may be used in any circumstances where specific degradation of uracil-DNA is required. Such applications that require the protein enzyme uracil-DNA nuclease as contained in the amino acid sequence constituted in SEQ ID NO:1, or SEQ ID NO:2, or SEQ ID NO:3, or SEQ ID NO:4, SEQ ID NO:5. are intended to be encompassed in the present invention. Due to the equivalent base-pairing capabilities of uracil and thymine, uracil-DNA and normal DNA may encode equivalent genetic information, if uracil is present only at thymine-replacing sites. One example for a useful application of uracil-DNA nuclease concerns biosafety.

Biosafety application of uracil-DNA nuclease. For specific degradation of recombinant DNA encoding potentially not desired, or even harmful, genetic information produced in vitro under laboratory circumstances, the use of uracil-DNA nuclease provides a rather simple and straightforward solution. Recombinant DNA is frequently produced in the laboratory and its escape from the laboratory is not always strictly ensured. Those skilled in the art will recognize, or be able to ascertain using no more than routine experimentation, it will be possible, using general methods known in the art, to produce all recombinant DNA with a significant uracil content. For in vitro production, inclusion of dUTP into the polymerase reactions will ensure the production of uracil-DNA. For cellular experiments in E. coli, the use of dut-ung-mutant E. coli strain will ensure the production of uracil-DNA. These uracil-DNA species will be equivalent in genetic coding to normal DNA of the same sequence where the deoxyuridine residue is replaced by thymine. Recombinant DNA with uracil content will be degradable by uracil-DNA nuclease with high efficiency, while normal DNA will not be degraded. Recombinant DNA will be therefore prevented from escaping the laboratory by the use of uracil-DNA nuclease.

Molecular diagnostics application of uracil-DNA nuclease. To ascertain the presence of a specific mutation in e.g. human or other genome, uracil-DNA nuclease may be applied. Some defined mutations in the human genome are well known to be involved in several pathogenic conditions. Molecular diagnostics techniques are known in the art to recognize such mutations by sequencing. With the use of uracil-DNA nuclease, a simple method with ease of use may be designed that does not require sequencing. In such a method, DNA oligomers hybridizing to the mutated site may be synthesized containing deoxyuridine residues. The oligomers may be labelled at the 5′ and 3′ end, with flurescent dyes capable of flurescence energy transfer. The labelling may be designed in such a way that within the intact oligomer, fluorescence is quenched due to the short distance between the two fluorescent labels as defined by the length of the oligonucleotide. However, fluorescence may be significantly increased if the distance between the two labels increase due to cleavage of the oligonucleotide. Such a deoxyuridine residue-containing double-labelled oligonucletide with its controlled sequence that is 100% complementary to the sequence containing the mutation to be investigated may hybridize to a site if it contains the mutation to be investigated. If the mutation is not present, hybridization will not occur. After the hydridization experiment, the hybrid double stranded DNA may be separated from single stranded DNA by usual methods known in the art. The double stranded DNA may then be treated with uracil-DNA nuclease that will result in cleavage of the uracil-containing oligonucleotide strand and therefore fluorescence intensity will increase. Increment in flurescent intensity, as detected by methods known in the art, will then reflect the existence of the mutation.

Persons skilled in the art will recognize, or will be able to design using no more than routine experimentation, many equivalents of the specific embodiments of the invention described herein. Such equivalents are intended to be included in the present invention.

SEQ ID NO:1

M I K C H M P S S W R R L R K I S R I L A L T G S
R Q I L T Q V L A T K G A A M A E G D S
K F G F K D M E K A L E T L K L L E S H
D M Q Y R K L T V R G L L G R A K R V L
T M T K A E E K L K N I N A A I G V F E
K W L E E N G G G A S S K N A K T E S E
D K V E T V P G L G F K D K A A A E A T
L S I L A E R D P D Y Q R L A I K G L I
G S S K R V L S G T K N E D K I T A I K
E G V Q V L E D F L E K F E A E N R I K
D N R A Y L P L A V V T K L P D P K D E
L A K E F L E A Y G G S K A K G N Y K H
L R T M F P K T D E K T S W D I V R N R
Q L S K L L E Q I K S E E A K L F D A E
T G A P T D L H L Q L I H W A Y S P Q P
D K L K Q Y I E K L A K K T P E K R K Q
E S S S S A S D S S A T S Q D S D G E D
K P K R K K K R E E

SEQ ID NO:2

M S E G E S K F G F K D M E K A L E T L
K L L E D H D M Q Y R K L T V R G L L G
R A K R V L T L T K A E E K L K N I N E
A I G V F E K W L E D N G G G A S S K N
A K T D N E D K V E T V P G L G F K D K
A A A E A T L S I L A E R D P D Y Q R L
A I K G L I G S S K R V L S G T K N E D
K I N S I K E G V Q V L E D F L E K F E
A E N R I K E N R A Y L A Y A V V S K L
P E P K E D L S V E F L A A Y G G S K A
K G N Y K H L R T M Y P K E D D T T S W
D I V R N R Q I A K L L E Q I K S E E A
K L F D S E S G E P T D L H L Q L I H W
A Y T P Q P D K V K A Y V E K L A K K T
P Q K R K P E

SEQ ID NO:3

M A K E E S K Y G F K D K A
K A E E S L E L L K S E D H K Y Q L L T
V R G L I G R A K R V L T L T K A E D K
I N N I K A A I E T F E Q W L E A N S S
S S T K N A K P K D A E D K V E T V P G
L G F K D K Q A A E Q T L S I L E G R D
P D Y Q K L A I K G L I G S A K R V I P
A T K N E E K L S S I K Q A V A L F E D
F L D R F D R E E R G K Q N M P Y L S I
D L I R Q L P A P Q G E Q S D K L A V E
F L A C Y E T Q A K G N Y K H L R T K A
P K D P G S K T W D I V R N A K L Q A L
K P D S S V K L F D Q D G K P T E L H L
Q M V Q W A Y S P Q V E K L K S Y A N S
L A S S G K S T T P S R K R T H S S S S
S S E Q E S K A K E S K K D R K K S K K

SEQ ID NO:4

K S T E P E E S V M G F K D K Q K A L D
T L K A L D G R D I S Y Q Y H V I A S F
V S R A K R T L Q I T R D E E K L A N I
R E A L K V F E D W L A N Y K E N N R S
K E N L A Y L P I E T I K G F K S L A K
N G L G F K D K E K A L Q T I K L L E G
R D L N Y Q Y H A I S G L V K R A E R V
I S C T K D E Q K L K N I K E A V E V F
D N W I T D F K V N G R A K M N F D Y L
S V D L V R S Y K P L A D K Y K I E D N
G F L K A Y E E V D G D Y K K L R N V Q
V P D S S I T W D I E R N K N L Q N V V
D R V K E Q K K W F E T D G E F E D L P
T E G H I R C I M W A Y S H D A G K L K
K L L P T L A E K L K S

SEQ ID NO:5

M L Y K N E R S V K L T I G R V D S D N
R Q K F S D R P I A Q Y A S D F N V E V
F I V L C F R T M G K D D K E D T G F G
F K D K A K A E D T L R L L E E H D L N
Y R R L T V R G L L G R A K R V L S M T
K A E E K I K N I K E A M E V F E N W L
A D L D K N K E Q K E K P E K K E K K D
T V P G L G F K D K S A A E G T L K V L
D G R D P D Y Q R L A V K G L I G R A K
R V L T C T R D E T K V S N I K E A I T
V F E K F L D D F E S L H L S K E N N P
Y L S L G V V R A A E Q L A G E S K S F
I A A Y S S V N G E Y K R L R T V E E S
E G G L T W D I V R N N A L K P L K A T
H A E A K L F N E E G E P T P E H L E L
I L W A Y S P E A A R L K K C L P E V E
T E V S R K R R S S A Q E E S P A K K K
K D

SEQ ID NO:6

atgattaagtgccatatgccgtcgagttggagacggctacgcaaaatcag
tcgtattctagcgctgacaggaagcagacagatactcacccaagtattag
caaccaaaggagcagcaatggcagagggagattcgaaatttggcttcaag
gacatggagaaggcgttagagacgttgaagctgctggagagtcacgacat
gcagtatcgcaagctgacggtgcgcggtttgcttggccgggccaaaagag
tcctgacaatgaccaaggcggaggagaagctgaagaacatcaatgcggcc
attggagtctttgaaaagtggctggaggagaatggcggaggggcgtccag
caagaatgccaagacagagagcgaggacaaggtggagacggtgccgggat
tgggattcaaggacaaggctgctgcggaggcaacgctgagcattttggcg
gaacgagatccggactaccagaggttggccatcaagggattgattggcag
ctccaagcgtgtcctgtcaggcaccaagaacgaggacaagatcacggcca
taaaggagggagtccaggtacttgaggatttcctcgaaaagttcgaggcc
gagaatcgtatcaaggacaatcgagcatacttgccactcgccgtggtcac
caaactgcccgatcccaaagatgagttggctaaggagtttctcgaagcct
atggcggctccaaggccaagggtaactacaagcacctgcgcacaatgttc
cccaaaacggatgaaaagaccagctgggatattgtgcgcaatcgtcagct
gtccaagttgctggagcagattaagagtgaggaggccaagctcttcgatg
cagagaccggagcacccaccgacctgcacctgcagttgatccactgggca
tacagtccgcagccggacaagctgaagcagtacatcgaaaagctggccaa
gaagacgcccgaaaagcgcaagcaggagagcagcagcagtgccagcgatt
ccagtgccaccagccaggattccgatggcgaggataagcccaaaaggaag
aagaagagggaggag

SEQ ID NO:7

atgtcggagggagagtcaaagtttggtttcaaggacatggagaaggccct
ggagacgctgaagctgctggaggatcatgacatgcagtaccgaaagctga
ccgtgcgcggtctccttggacgcgccaagcgagtgctgaccttgaccaag
gcggaggagaagctgaagaacatcaatgaggcgattggcgtgttcgagaa
atggctggaggataatggcggcggggcgtccagcaagaacgccaagactg
acaacgaggataaggtggagaccgtgcccggactgggcttcaaggacaag
gcggcggcggaggcgacgctgagcattctggcggagcgtgacccggacta
ccagaggctggccatcaagggattgattggcagctccaagcgagtgctgt
ccggcaccaagaacgaggacaagatcaattccatcaaggagggagtccag
gtgctggaggatttcctggagaagttcgaggccgagaaccgcatcaagga
gaatcgcgcctacttggcctatgccgtcgtgtccaagctgccagagccca
aagaggatctgtccgtcgagttcctggctgcctacggcggctccaaggcc
aagggcaactacaagcacctgcgcaccatgtaccccaaggaggacgacac
caccagctgggacattgtgcgcaatcgccagatagccaagctgctggagc
agatcaagagcgaggaggccaagctgttcgactcggagtcgggcgagccc
acagatctccacttgcagctgatccactgggcctacacgccccagccgga
caaggtgaaggcctatgtggagaagctggccaagaagacgccgcagaagc
gcaagccggag

SEQ ID NO:8

atggcaaaggaagaatcaaagtacggcttcaaggataaggcc
aaggccgaggagtcgctggagctgctgaagagcgaggatcacaagtacca
gctgctgacggtgcgcggtctgatcggacgggcgaagcgtgtgctgacat
tgacgaaggctgaggacaagataaacaacataaaggctgcgatcgaaacg
ttcgagcagtggctggaagcaaacagctcctccagcaccaaaaacgcaaa
gccgaaggatgcagaagacaaggtggaaactgtgccagggttgggtttca
aggacaagcaggcggctgaacaaacgctgagcatcctagaagggcgcgat
cccgattatcagaagctagcaatcaagggactgatcggtagcgcaaagcg
cgttatccctgccaccaagaacgaggagaagctaagctcgatcaagcaag
cggtggcactgtttgaagactttctcgatcggttcgatcgcgaggagcgg
ggcaagcaaaacatgccgtacctttcgatcgacttgatacgtcaactgcc
cgcaccgcagggggagcagtcggacaagctggcagtggaatttctcgcct
gctatgaaacgcaggccaaaggcaactacaaacatttgcgcaccaaagca
cccaaggacccaggctcgaagacgtgggacattgtgcgaaatgcgaaact
gcaggcactgaaaccggacagcagtgtgaagttattcgatcaggacggca
agcctaccgagctgcacttgcagatggtacagtgggcgtacagcccacag
gtggagaagctgaagagctatgcgaacagtttggcgagcagtggcaaatc
aacaacaccgtccaggaaacgaacgcactcttccagctcatcttcggagc
aggagtcaaaggcgaaggagagtaagaaggatcgcaaaaagtcgaagaaa

SEQ ID NO:9

aaatcaacggaaccggaagaatccgtgatgggtttcaaggataagcaaaa
ggccttggacacgctgaaagctctggacggccgagatatcagttatcagt
accatgtgattgctagttttgtgagccgcgcgaaaagaacgttgcagatt
acaagggacgaggaaaagctggccaatatacgagaagctttgaaagtgtt
cgaggattggctcgctaactacaaagagaacaatcgcagtaaagaaaacc
tcgcttacttgccaatcgagactataaaaggcttcaagagccttgccaaa
aacggactcggttttaaagataaagagaaggctttgcagactatcaagtt
actggaaggtagggatctaaattatcagtaccacgcgatctctggccttg
tgaaaagagctgagcgagtgatatcgtgcacaaaggacgagcaaaaactc
aagaacataaaagaagctgtggaagtgttcgacaattggattacggattt
caaggtaaatggccgggcaaagatgaatttcgattatttatccgttgatt
tggtacgatcctacaaaccgttggcggacaagtacaaaatcgaagataat
ggatttcttaaagcgtacgaagaagtggatggagattataagaaattgag
aaacgttcaagttccagattcgagtattacctgggatatagagaggaata
agaatcttcaaaatgtcgtagatcgtgtcaaagaacaaaagaaatggttc
gagacggatggtgagttcgaagatttacccaccgaaggacacattcgatg
tataatgtgggcttacagtcacgatgcaggtaaattgaaaaaacttttgc
ctacgttagctgaaaagttaaaatcg

SEQ ID NO:10

atgttatataaaaacgaaagatctgtcaagttaactataggtagagttga
ttcagacaatcgtcaaaaattctctgatcgtccgatagcacagtatgcat
ctgactttaatgtggaagtttttattgttttgtgtttcagaacaatgggc
aaagatgataaagaagatacaggatttggattcaaggacaaggcgaaggc
ggaggacacgctgcggctcctggaggagcacgacctgaactacaggaggc
tgacagtgagaggacttctcggtagagcgaagagggttctgtcaatgacg
aaagcagaagagaaaatcaaaaacatcaaagaggccatggaggtgttcga
gaactggctcgcggacctcgacaagaacaaggagcaaaaagagaagcccg
aaaagaaagagaagaaagacactgtgccgggcctaggcttcaaggataag
tctgccgcagaagggaccctcaaagtgttggacgggagagacccggatta
ccagagactggccgtcaagggccttatagggagagccaagcgggtgttga
cttgcacccgagatgagactaaagtatcgaacatcaaggaggccataacg
gtcttcgagaagttcctcgacgacttcgagagcttgcatctgagcaaaga
gaacaacccgtacctaagtctcggcgtggtgcgggccgccgagcagctgg
ccggggagagcaaatcgttcatagccgcctactcctccgtcaatggagaa
tacaagaggctgaggaccgtggaggagtcggaggggggcctcacttggga
catcgttaggaacaacgcgctcaaaccgctcaaagccacgcatgctgagg
cgaagctgttcaacgaagaaggcgaaccgaccccggaacatttagagtta
atactgtgggcgtactcaccggaggcggcccgcctcaagaagtgccttcc
cgaggtggaaacagaagttagccggaagagaagaagcagcgcgcaagaag
agtctccggctaaaaagaagaaggat

What is claimed is:

1. An isolated polypeptide comprising an amino acid sequence which shows at least 90% identity to any of the amino acid sequences selected from the group consisting of SEQ ID NO:1, SEQ ID NO:2, SEQ ID NO:3, SEQ ID NO:4, and SEQ ID NO:5.

2. The isolated polypeptide of claim 1, wherein the polypeptide is a polypeptide present in any of the following organisms: Drosophila melanogaster, or Drosophila pseudoobscura, or Anapheles gambiae, or Apis mellifera, or Bombyx mori.

3. An isolated nucleic acid sequence which shows at least 90% identity to any of the nucleic acid sequences selected from the group consisting of SEQ ID NO:6, SEQ ID NO:7, SEQ ID NO:8, SEQ ID NO:9, and SEQ ID NO:10.

4. A recombinant vector containing a nucleic acid sequence according to claim 3.

5. The recombinant vector of claim 4 which is an expression vector.

6. The recombinant vector of claim 5 wherein the nucleic acid sequence is linked to a second nucleic acid sequence encoding a heterologous amino acid sequence.

7. An isolated polypeptide comprising at least 55 consecutive amino acid residues of SEQ ID NO:1 and which has at least one bioactivity of the uracil-DNA nuclease enzyme; wherein the bioactivity is selected from the group consisting of:
(a) binding to DNA
(b) cleaving uracil-substituted DNA, wherein thymine bases are replaced by uracil bases in any given sequence constraint, but not cleaving normal DNA.

8. A recombinant vector containing a nucleic acid sequence encoding the amino acid sequence according to claim 7.

9. The recombinant vector of claim 8 which is an expression vector.

10. The recombinant vector of claim 9 wherein the nucleic acid sequence is linked to a second nucleic acid sequence encoding a heterologous amino acid sequence.

11. The recombinant vector of claim 5 wherein the heterologous amino acid sequence is an affinity purification tag sequence or a secretion signal sequence.

12. A host cell transformed with the recombinant vector of claim 4.

13. The transformed host cell of claim 12 which is an E. coli host cell.

14. A host cell transformed with the recombinant vector of claim 5.

15. The transformed host cell of claim 14 which is an E. coli host cell.

16. A process for obtaining a recombinant uracil-DNA nuclease enzyme protein, the process comprising the following steps: (a) culturing the transformed host cell of claim 12 in culture medium under conditions inducing expression of the recombinant uracil-DNA nuclease by the transformed host cell; (b) lysing host cells to produce a cell lysate comprising the recombinant uracil-DNA nuclease enzyme protein and other materials; (c) performing chromatography on an affinity column corresponding to an affinity purification tag present on the recombinant uracil-DNA nuclease enzyme protein to obtain a fraction enriched in the uracil-DNA nuclease enzyme protein; (d) performing size exclusion chromatography to obtain the purified uracil-DNA enzyme protein.

17. The process of claim 16 wherein the affinity tag is maltose binding protein or a polyhistidine peptide.

18. The process of claim 16 wherein the chromatographic step in claim 16 is replaced by ion exchange chromatography.

19. A process for specific cleavage of uracil-DNA by the enzyme protein of amino acid sequence according to claim 1.

20. A process for specific cleavage of uracil-DNA by the enzyme protein comprising the amino acid sequence according to claim 7.


SEQUENCE LISTING (10 sequences)

SEQ ID NO: 1 - protein, 355 aa, Drosophila melanogaster
MIKCHMPSSWRRLRKISRILALTGSRQILTQVLATKGAAMAEGDSKFGFKDMEKALETLKLLESHDMQYRKLTVRGLLGRAKRVLTMTKAEEKLKNINAAIGVFEKWLEENGGGASSKNAKTESEDKVETVPGLGFKDKAAAEATLSILAERDPDYQRLAIKGLIGSSKRVLSGTKNEDKITAIKEGVQVLEDFLEKFEAENRIKDNRAYLPLAVVTKLPDPKDELAKEFLEAYGGSKAKGNYKHLRTMFPKTDEKTSWDIVRNRQLSKLLEQIKSEEAKLFDAETGAPTDLHLQLIHWAYSPQPDKLKQYIEKLAKKTPEKRKQESSSSASDSSATSQDSDGEDKPKRKKKREE

SEQ ID NO: 2 - protein, 287 aa, Drosophila pseudoobscura
MSEGESKFGFKDMEKALETLKLLEDHDMQYRKLTVRGLLGRAKRVLTLTKAEEKLKNINEAIGVFEKWLEDNGGGASSKNAKTDNEDKVETVPGLGFKDKAAAEATLSILAERDPDYQRLAIKGLIGSSKRVLSGTKNEDKINSIKEGVQVLEDFLEKFEAENRIKENRAYLAYAVVSKLPEPKEDLSVEFLAAYGGSKAKGNYKHLRTMYPKEDDTTSWDIVRNRQIAKLLEQIKSEEAKLFDSESGEPTDLHLQLIHWAYTPQPDKVKAYVEKLAKKTPQKRKPE

SEQ ID NO: 3 - protein, 314 aa, Anopheles gambiae
MAKEESKYGFKDKAKAEESLELLKSEDHKYQLLTVRGLIGRAKRVLTLTKAEDKINNIKAAIETFEQWLEANSSSSTKNAKPKDAEDKVETVPGLGFKDKQAAEQTLSILEGRDPDYQKLAIKGLIGSAKRVIPATKNEEKLSSIKQAVALFEDFLDRFDREERGKQNMPYLSIDLIRQLPAPQGEQSDKLAVEFLACYETQAKGNYKHLRTKAPKDPGSKTWDIVRNAKLQALKPDSSVKLFDQDGKPTELHLQMVQWAYSPQVEKLKSYANSLASSGKSTTPSRKRTHSSSSSSEQESKAKESKKDRKKSKK

SEQ ID NO: 4 - protein, 292 aa, Apis mellifera
KSTEPEESVMGFKDKQKALDTLKALDGRDISYQYHVIASFVSRAKRTLQITRDEEKLANIREALKVFEDWLANYKENNRSKENLAYLPIETIKGFKSLAKNGLGFKDKEKALQTIKLLEGRDLNYQYHAISGLVKRAERVISCTKDEQKLKNIKEAVEVFDNWITDFKVNGRAKMNFDYLSVDLVRSYKPLADKYKIEDNGFLKAYEEVDGDYKKLRNVQVPDSSITWDIERNKNLQNVVDRVKEQKKWFETDGEFEDLPTEGHIRCIMWAYSHDAGKLKKLLPTLAEKLKS

SEQ ID NO: 5 - protein, 342 aa, Bombyx mori
MLYKNERSVKLTIGRVDSDNRQKFSDRPIAQYASDFNVEVFIVLCFRTMGKDDKEDTGFGFKDKAKAEDTLRLLEEHDLNYRRLTVRGLLGRAKRVLSMTKAEEKIKNIKEAMEVFENWLADLDKNKEQKEKPEKKEKKDTVPGLGFKDKSAAEGTLKVLDGRDPDYQRLAVKGLIGRAKRVLTCTRDETKVSNIKEAITVFEKFLDDFESLHLSKENNPYLSLGVVRAAEQLAGESKSFIAAYSSVNGEYKRLRTVEESEGGLTWDIVRNNALKPLKATHAEAKLFNEEGEPTPEHLELILWAYSPEAARLKKCLPEVETEVSRKRRSSAQEESPAKKKKD

SEQ ID NO: 6 - DNA, 1065 bp, Drosophila melanogaster
atgattaagtgccatatgccgtcgagttggagacggctacgcaaaatcagtcgtattctagcgctgacaggaagcagacagatactcacccaagtattagcaaccaaaggagcagcaatggcagagggagattcgaaatttggcttcaaggacatggagaaggcgttagagacgttgaagctgctggagagtcacgacatgcagtatcgcaagctgacggtgcgcggtttgcttggccgggccaaaagagtcctgacaatgaccaaggcggaggagaagctgaagaacatcaatgcggccattggagtctttgaaaagtggctggaggagaatggcggaggggcgtccagcaagaatgccaagacagagagcgaggacaaggtggagacggtgccgggattgggattcaaggacaaggctgctgcggaggcaacgctgagcattttggcggaacgagatccggactaccagaggttggccatcaagggattgattggcagctccaagcgtgtcctgtcaggcaccaagaacgaggacaagatcacggccataaaggagggagtccaggtacttgaggatttcctcgaaaagttcgaggccgagaatcgtatcaaggacaatcgagcatacttgccactcgccgtggtcaccaaactgcccgatcccaaagatgagttggctaaggagtttctcgaagcctatggcggctccaaggccaagggtaactacaagcacctgcgcacaatgttccccaaaacggatgaaaagaccagctgggatattgtgcgcaatcgtcagctgtccaagttgctggagcagattaagagtgaggaggccaagctcttcgatgcagagaccggagcacccaccgacctgcacctgcagttgatccactgggcatacagtccgcagccggacaagctgaagcagtacatcgaaaagctggccaagaagacgcccgaaaagcgcaagcaggagagcagcagcagtgccagcgattccagtgccaccagccaggattccgatggcgaggataagcccaaaaggaagaagaagagggaggag

SEQ ID NO: 7 - DNA, 861 bp, Drosophila pseudoobscura
atgtcggagggagagtcaaagtttggtttcaaggacatggagaaggccctggagacgctgaagctgctggaggatcatgacatgcagtaccgaaagctgaccgtgcgcggtctccttggacgcgccaagcgagtgctgaccttgaccaaggcggaggagaagctgaagaacatcaatgaggcgattggcgtgttcgagaaatggctggaggataatggcggcggggcgtccagcaagaacgccaagactgacaacgaggataaggtggagaccgtgcccggactgggcttcaaggacaaggcggcggcggaggcgacgctgagcattctggcggagcgtgacccggactaccagaggctggccatcaagggattgattggcagctccaagcgagtgctgtccggcaccaagaacgaggacaagatcaattccatcaaggagggagtccaggtgctggaggatttcctggagaagttcgaggccgagaaccgcatcaaggagaatcgcgcctacttggcctatgccgtcgtgtccaagctgccagagcccaaagaggatctgtccgtcgagttcctggctgcctacggcggctccaaggccaagggcaactacaagcacctgcgcaccatgtaccccaaggaggacgacaccaccagctgggacattgtgcgcaatcgccagatagccaagctgctggagcagatcaagagcgaggaggccaagctgttcgactcggagtcgggcgagcccacagatctccacttgcagctgatccactgggcctacacgccccagccggacaaggtgaaggcctatgtggagaagctggccaagaagacgccgcagaagcgcaagccggag

SEQ ID NO: 8 - DNA, 942 bp, Anopheles gambiae
atggcaaaggaagaatcaaagtacggcttcaaggataaggccaaggccgaggagtcgctggagctgctgaagagcgaggatcacaagtaccagctgctgacggtgcgcggtctgatcggacgggcgaagcgtgtgctgacattgacgaaggctgaggacaagataaacaacataaaggctgcgatcgaaacgttcgagcagtggctggaagcaaacagctcctccagcaccaaaaacgcaaagccgaaggatgcagaagacaaggtggaaactgtgccagggttgggtttcaaggacaagcaggcggctgaacaaacgctgagcatcctagaagggcgcgatcccgattatcagaagctagcaatcaagggactgatcggtagcgcaaagcgcgttatccctgccaccaagaacgaggagaagctaagctcgatcaagcaagcggtggcactgtttgaagactttctcgatcggttcgatcgcgaggagcggggcaagcaaaacatgccgtacctttcgatcgacttgatacgtcaactgcccgcaccgcagggggagcagtcggacaagctggcagtggaatttctcgcctgctatgaaacgcaggccaaaggcaactacaaacatttgcgcaccaaagcacccaaggacccaggctcgaagacgtgggacattgtgcgaaatgcgaaactgcaggcactgaaaccggacagcagtgtgaagttattcgatcaggacggcaagcctaccgagctgcacttgcagatggtacagtgggcgtacagcccacaggtggagaagctgaagagctatgcgaacagtttggcgagcagtggcaaatcaacaacaccgtccaggaaacgaacgcactcttccagctcatcttcggagcaggagtcaaaggcgaaggagagtaagaaggatcgcaaaaagtcgaagaaa

SEQ ID NO: 9 - DNA, 876 bp, Apis mellifera
aaatcaacggaaccggaagaatccgtgatgggtttcaaggataagcaaaaggccttggacacgctgaaagctctggacggccgagatatcagttatcagtaccatgtgattgctagttttgtgagccgcgcgaaaagaacgttgcagattacaagggacgaggaaaagctggccaatatacgagaagctttgaaagtgttcgaggattggctcgctaactacaaagagaacaatcgcagtaaagaaaacctcgcttacttgccaatcgagactataaaaggcttcaagagccttgccaaaaacggactcggttttaaagataaagagaaggctttgcagactatcaagttactggaaggtagggatctaaattatcagtaccacgcgatctctggccttgtgaaaagagctgagcgagtgatatcgtgcacaaaggacgagcaaaaactcaagaacataaaagaagctgtggaagtgttcgacaattggattacggatttcaaggtaaatggccgggcaaagatgaatttcgattatttatccgttgatttggtacgatcctacaaaccgttggcggacaagtacaaaatcgaagataatggatttcttaaagcgtacgaagaagtggatggagattataagaaattgagaaacgttcaagttccagattcgagtattacctgggatatagagaggaataagaatcttcaaaatgtcgtagatcgtgtcaaagaacaaaagaaatggttcgagacggatggtgagttcgaagatttacccaccgaaggacacattcgatgtataatgtgggcttacagtcacgatgcaggtaaattgaaaaaacttttgcctacgttagctgaaaagttaaaatcg

SEQ ID NO: 10 - DNA, 1026 bp, Bombyx mori
atgttatataaaaacgaaagatctgtcaagttaactataggtagagttgattcagacaatcgtcaaaaattctctgatcgtccgatagcacagtatgcatctgactttaatgtggaagtttttattgttttgtgtttcagaacaatgggcaaagatgataaagaagatacaggatttggattcaaggacaaggcgaaggcggaggacacgctgcggctcctggaggagcacgacctgaactacaggaggctgacagtgagaggacttctcggtagagcgaagagggttctgtcaatgacgaaagcagaagagaaaatcaaaaacatcaaagaggccatggaggtgttcgagaactggctcgcggacctcgacaagaacaaggagcaaaaagagaagcccgaaaagaaagagaagaaagacactgtgccgggcctaggcttcaaggataagtctgccgcagaagggaccctcaaagtgttggacgggagagacccggattaccagagactggccgtcaagggccttatagggagagccaagcgggtgttgacttgcacccgagatgagactaaagtatcgaacatcaaggaggccataacggtcttcgagaagttcctcgacgacttcgagagcttgcatctgagcaaagagaacaacccgtacctaagtctcggcgtggtgcgggccgccgagcagctggccggggagagcaaatcgttcatagccgcctactcctccgtcaatggagaatacaagaggctgaggaccgtggaggagtcggaggggggcctcacttgggacatcgttaggaacaacgcgctcaaaccgctcaaagccacgcatgctgaggcgaagctgttcaacgaagaaggcgaaccgaccccggaacatttagagttaatactgtgggcgtactcaccggaggcggcccgcctcaagaagtgccttcccgaggtggaaacagaagttagccggaagagaagaagcagcgcgcaagaagagtctccggctaaaaagaagaaggat